(19) United States
(12) Patent Application Publication
(10) Pub. No.: US 2022/0327395 A1
(43) Pub. Date: Oct. 13, 2022

(54) DATA ANALYZING APPARATUS, METHOD, AND PROGRAM

(71) Applicant: NIPPON TELEGRAPH AND TELEPHONE CORPORATION, Tokyo (JP)

(72) Inventors: Tae SATO, Musashino-shi, Tokyo (JP); Akihiro CHIBA, Musashino-shi, Tokyo (JP); Tomoki WATANABE, Musashino-shi, Tokyo (JP); Shozo AZUMA, Musashino-shi, Tokyo (JP); Takuya INDO, Musashino-shi, Tokyo (JP)

(73) Assignee: NIPPON TELEGRAPH AND TELEPHONE CORPORATION, Tokyo (JP)

(21) Appl. No.: 17/639,203

(22) PCT Filed: Sep. 3, 2019
(86) PCT No.: PCT/JP2019/034604

§ 371 Date: Feb. 28, 2022

(51) Int. Cl. G06N 5/02

(52) U.S. Cl. CPC G06N 5/022

(57) ABSTRACT

A data analysis apparatus according to the embodiment includes factor data collection means for collecting factor data assumed to affect to-be-predicted data serving as an objective variable; and generation means for calculating a corrected partial differential value of a change in a value of the factor data based on a degree of influence of a factor on the objective variable, which is a characteristic of the change in the value of the factor data, for each type of the factor data collected by the factor data collection means and generating an explanatory variable based on the corrected partial differential value.

### TECHNICAL FIELD

An embodiment of the present invention relates to a data analysis apparatus, method, and program.

### BACKGROUND ART

There is a technique that allows a ratio scale of quantitative data such as area and a nominal scale of qualitative data such as a land category to be inputted as explanatory variables and calculates contribution of each of the explanatory variables to an objective variable, which is to-be-predicted data, for example, using a land price as the objective variable. Note that the qualitative data is expressed by a one-hot vector in which only appropriate elements are assigned 1 and other elements are assigned 0 (see, for example, Non-Patent Literature 1).

### CITATION LIST

### Non-Patent Literature

Non-Patent Literature 1: “A Technique for Estimating Land Prices Using Multiple Regression Analysis,” Okayama University, DEIM Forum 2018 H5-3, on the Internet at http://db-event.jpn.org/deim2018/data/papers/195.pdf

### SUMMARY OF THE INVENTION

### Technical Problem

Whereas in Non-Patent Literature 1 described above, the ratio scale of quantitative data and the nominal scale of qualitative data are treated as explanatory variables, it will sometimes be desired to conduct regression analysis by taking into consideration an interval scale of quantitative data such as temperature (centigrade temperature) and a subjective fatigue degree as well as an ordinal scale of qualitative data such as subjective order.

In this case, a conceivable method involves using an interval scale or an ordinal scale as an explanatory variable by expressing the scale by a one-hot vector depending on whether there are appropriate numerical values among individual values of the interval scale and ordinal scale or whether appropriate condition ranges have been specified. However, the one-hot vector, in which each factor is expressed as an independent factor, does not take into consideration any change in the value of the factor, such as a difference in temperature or a change in fatigue degree.

Therefore, even if actually an amount of change such as a difference between whether the amount of change is 1 or 2, or values before and after the change such as a difference between whether the change is made from 4 to 3 or from 2 to 3 contribute to explanation of an objective variable, the factors cannot be extracted and accuracy of data analysis conducted using the explanatory variable is insufficient.

The present invention has been made in view of the above circumstances and has an object to provide a data analysis apparatus, method, and program that can improve accuracy of data analysis conducted using explanatory variables.

### Means for Solving the Problem

A data analysis apparatus according to one aspect of the present invention comprises: factor data collection means for collecting factor data assumed to affect to-be-predicted data serving as an objective variable; and generation means for calculating a corrected partial differential value of a change in a value of the factor data based on a degree of influence of a factor on the objective variable, which represents a characteristic of the change in the value of the factor data, for each type of the factor data collected by the factor data collection means and generating an explanatory variable based on the corrected partial differential value.

A data analysis method according to another aspect of the present invention is performed by a data analysis apparatus, the method comprising: collecting factor data assumed to affect to-be-predicted data serving as an objective variable; and calculating a corrected partial differential value of a change in a value of the factor data based on a degree of influence of a factor on the objective variable, which is a characteristic of the change in the value of the factor data, for each type of the collected factor data and generating an explanatory variable based on the corrected partial differential value.

### Effects of the Invention

The present invention can improve accuracy of data analysis conducted using explanatory variables.

### BRIEF DESCRIPTION OF DRAWINGS

FIG. 1 is a diagram showing an exemplary hardware configuration of a contribution estimation apparatus according to an embodiment of the present invention.

FIG. 2 is a diagram showing an exemplary software configuration of the contribution estimation apparatus according to the embodiment of the present invention.

FIG. 3 is a diagram showing, in tabular form, an example of factor data registered in a factor data DB.

FIG. 4 is a diagram showing, in tabular form, an example of one-hot vector generation conditions stored in a one-hot vector generation condition DB.

FIG. 5 is a diagram showing, in tabular form, an example of one-hot vector data registered in a one-hot vector DB.

FIG. 6 is a diagram showing, in tabular form, an example of variation vector generation conditions stored in a variation vector generation condition DB.

FIG. 7 is a diagram showing, in tabular form, a first example of interval/ordinal scale variation vector generation functions stored in an interval/ordinal scale variation vector generation function DB.

FIG. 8 is a diagram explaining a first example of a transfer function.

FIG. 9 is a diagram showing, in tabular form, a first example of factor data on an interval scale, where the factor data is stored in the factor data DB.

FIG. 10 is a diagram showing, in tabular form, a first example of variation vectors stored in a variation vector DB.

FIG. 11 is a diagram explaining a second example of the transfer function.

FIG. 12 is a diagram showing, in tabular form, a third example of the factor data stored in the factor data DB.

FIG. 13 is a diagram showing, in tabular form, a second example of the interval/ordinal scale variation vector generation functions stored in the interval/ordinal scale variation vector generation function DB.

FIG. 14 is a diagram showing, in tabular form, a second example of the variation vectors stored in the variation vector DB.

FIG. 15 is a flowchart showing an example of processing procedures for determining a corrected partial differential value.

FIG. 16 is a diagram showing an example of various transfer functions.

FIG. 17 is a diagram showing, in tabular form, a third example of the interval/ordinal scale variation vector generation functions stored in the interval/ordinal scale variation vector generation function DB.

FIG. 18 is a diagram showing, in tabular form, an example of an objective variable stored in an objective variable DB.

FIG. 19 is a diagram showing, in tabular form, an example of weights calculated by regression analysis and stored in a weight DB.

FIG. 20 is a diagram showing an example of behavioral impact scores.

### DESCRIPTION OF EMBODIMENT

An embodiment of the present invention will be described below with reference to the drawings.

(Configuration)

(1) Hardware Configuration

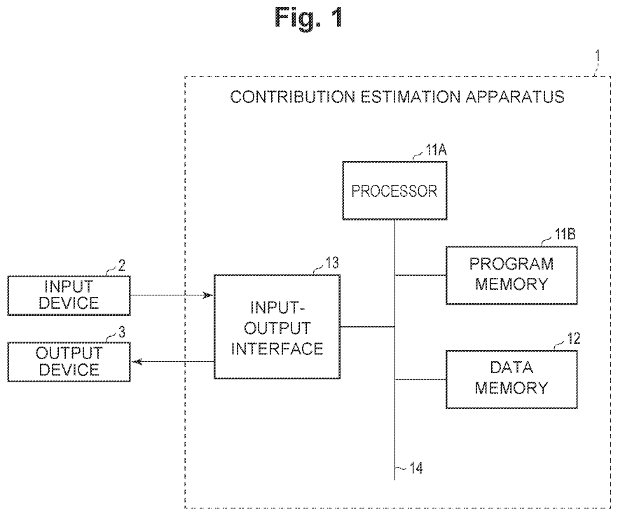

FIG. 1 is a block diagram showing an exemplary hardware configuration of a contribution estimation apparatus 1 according to an embodiment of the present invention.

The contribution estimation apparatus 1 is made up, for example, of a server computer or a personal computer, and includes a hardware processor 11A such as a CPU (Central Processing Unit). In the contribution estimation apparatus 1, a program memory 11B, a data memory 12, and an input-output interface 13 are connected to the hardware processor 11A via a bus 14.

An input device 2, such as a keyboard, and an output device 3 are attached to the contribution estimation apparatus 1. The input device 2 and the output device 3 can be connected to the input-output interface 13. The program memory 11B, which is a non-transitory tangible computer-readable storage medium, is made up of a combination of, for example, a nonvolatile memory, such as an HDD (Hard Disk Drive) or an SSD (Solid State Drive), which allows random access, and a nonvolatile memory such as a ROM. Programs needed in performing various control processes according to the embodiment are stored in the program memory 11B.

The data memory 12, which is a tangible computer-readable storage medium, is made up of a combination of, for example, a nonvolatile memory such as described above and a volatile memory such as a RAM (Random Access Memory). The data memory 12 is used to store various data acquired and created in the course of performing various processes.

(2) Software Configuration

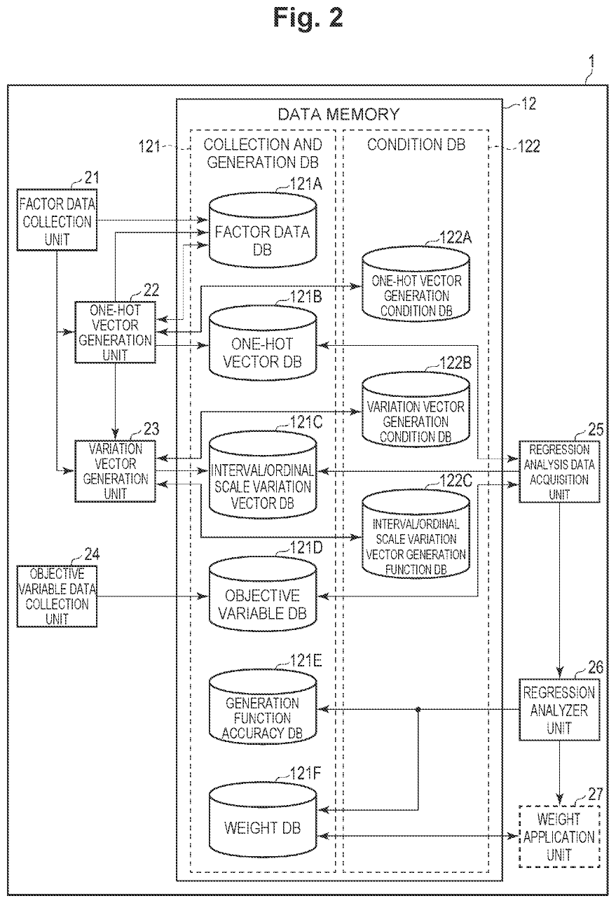

FIG. 2 is a diagram showing an exemplary software configuration of the contribution estimation apparatus 1 according to the embodiment of the present invention. In FIG. 2, the software configuration of the contribution estimation apparatus 1 is shown by being associated with the hardware configuration shown in FIG. 1.

As shown in FIG. 2, the contribution estimation apparatus 1 can be configured as a data analysis apparatus equipped with software-based processing functional components including a factor data collection unit 21, a one-hot vector generation unit 22, an interval/ordinal scale variation vector generation unit (also referred to as a variation vector generation unit) 23, an objective variable data collection unit 24, a regression analysis data acquisition unit 25, a regression analyzer unit 26, a weight application unit 27, a collection and generation DB (database) 121, and a condition DB 122.

The collection and generation DB 121 includes a factor data DB 121A, a one-hot vector DB 121B, a variation vector DB 121C, an objective variable DB 121D, a generation function accuracy DB 121E, and a weight DB 121F.

The condition DB 122 includes a one-hot vector generation condition DB 122A, a variation vector generation condition DB 122B, and an interval/ordinal scale variation vector generation function DB (also referred to as a variation vector generation function DB) 122C. It is assumed that various information is stored in advance in various components of the condition DB 122.

The collection and generation DB (database) 121 and the condition DB 122 in the contribution estimation apparatus 1 shown in FIG. 2 can be constructed of the data memory 12 shown in FIG. 1. However, these databases are not essential components of the contribution estimation apparatus 1, and may be provided, for example, in an external storage mediums such as a USB (Universal Serial Bus) memory or in a storage device such as a database server placed in the cloud.

Processing functional components in all the factor data collection unit 21, one-hot vector generation unit 22, interval/ordinal scale variation vector generation unit 23, objective variable data collection unit 24, regression analysis data acquisition unit 25, regression analyzer unit 26, weight application unit 27, collection and generation DB (database) 121, and condition DB 122 are implemented when the programs stored in the program memory 11B are read out and executed by the hardware processor 11A above. Note that some or all of the processing functional components may be implemented in various other forms including integrated circuits such as application specific integrated circuits (ASICs) or field-programmable gate arrays (FPGAs).

The contribution estimation apparatus 1 newly calculates quantitative data that reflects characteristics of changes (a degree of influence on objective variables) in factors assumed to affect the objective variables (when a scale type is interval scale or ordinal scale) and adds the calculated data to explanatory variables.

The present embodiment can improve accuracy of factor analysis when factor data explaining objective variables contains interval scale data of a subjective questionnaire or ordinal scale data and there are changes in the value of the factor data. Furthermore, the present embodiment makes it possible to estimate contribution of the changes to the objective variables.

Components of the contribution estimation apparatus 1 will be described in detail below.

(1) Factor Data Collection Unit

The factor data collection unit 21 collects data of predetermined factors assumed to affect objective variables at a specified frequency such as at a specified time, each time data is acquired, or the like. The factor data collection unit 21 registers the collected data in the factor data DB 121A by associating the data with the current date and time recorded by a built-in timer.

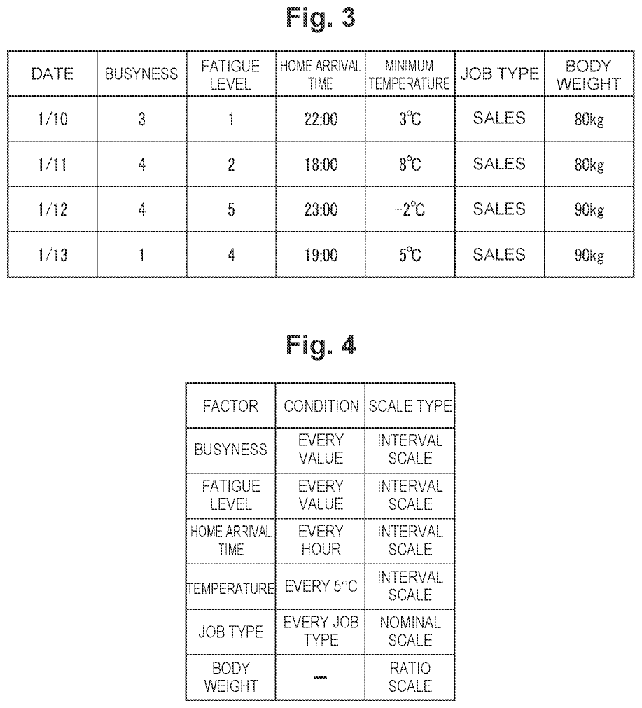

FIG. 3 is a diagram showing, in tabular form, an example of factor data registered in the factor data DB 121A.

For example, when an objective variable is “whether running is to be done,” it is assumed that factor data are “busyness of user,” “fatigue level of user,” “home arrival time,” “temperature (e.g., minimum temperature),” “job type,” and “body weight” shown in FIG. 3. Although not shown in FIG. 3, examples of factor data include “mental leeway.”

“Busyness of user” and “fatigue level of user” are collected just when entered by the user via the input device 2. “Home arrival time,” and “body weight” are collected, for example, at the end of the day (e.g., at 23:59). “Temperature” is collected, for example, at the start of the day (e.g., at 00:01). “Job type” is collected, for example, once a year.

By providing user identifiers, factor data on plural users may be collected.

(2) One-Hot Vector Generation Unit

FIG. 4 is a diagram showing, in tabular form, an example of one-hot vector generation conditions stored in the one-hot vector generation condition DB 122A.

As shown in FIG. 4, each factor in the factor data registered in the factor data DB 121A is registered in the one-hot vector generation condition DB 122A by being associated with a one-hot vector generation condition (condition) and a scale type. One-hot vector generation conditions include every value, every predetermined time interval, every predetermined temperature interval, and every job type. Scale types include interval scale, nominal scale, ratio scale, and ordinal scale. Note that although neither an ordinal scale nor a condition therefor is shown in the example of FIG. 4, if there is a factor on an ordinal scale, a one-hot vector generation condition and a scale type associated with the factor can be stored in the one-hot vector generation condition DB 122A.

With reference to the factor data DB 121A and the one-hot vector generation condition DB 122A, the one-hot vector generation unit 22 generates one-hot vector data by converting factor data into one-hot vectors. The one-hot vector generation unit 22 registers the generated one-hot vector data in the one-hot vector DB 121B.

If the generated one-hot vector data includes factor data, such as weight data, whose scale type is ratio scale, the one-hot vector generation unit 22 obtains final one-hot vector data by normalizing one-hot vector values of the factor data.

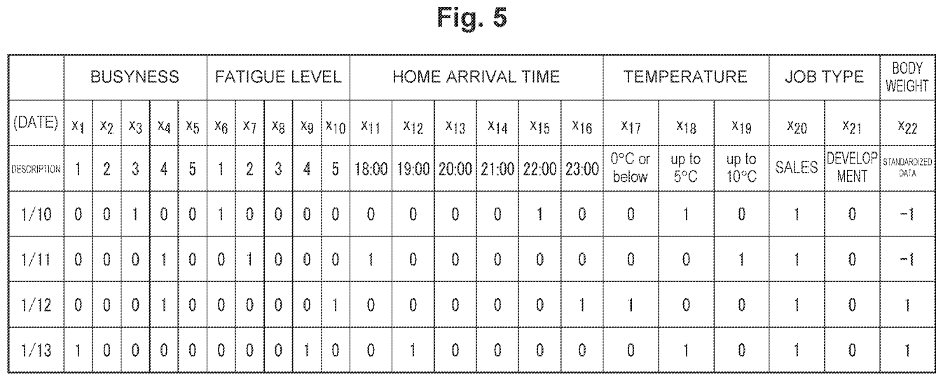

FIG. 5 is a diagram showing, in tabular form, an example of one-hot vector data registered in the one-hot vector DB 121B. FIG. 5 shows normalized one-hot vector data of each factor on each date.

(3) Variation Vector Generation Unit

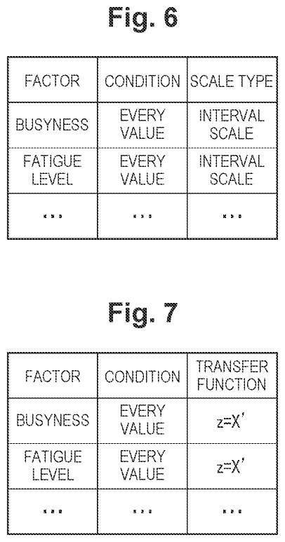

FIG. 6 is a diagram showing, in tabular form, an example of variation vector generation conditions stored in the variation vector generation condition DB 122B.

As shown in FIG. 6, when the factor data registered in the factor data DB 121A includes factor data on an interval scale or factor data on an ordinal scale, the variation vector generation condition (Condition in FIG. 6) and scale type of each factor on the scale are stored in the variation vector generation condition DB 122B by being associated with each other. Possible variation vector generation conditions include every value, every predetermined time interval, every predetermined temperature interval, and the like.

FIG. 7 is a diagram showing, in tabular form, a first example of variation vector generation functions stored in the interval/ordinal scale variation vector generation function DB 122C.

As shown in FIG. 7, of the variation vector generation functions stored in the interval/ordinal scale variation vector generation function DB 122C, a transfer function z is stored by being associated with the variation vector generation condition of each factor stored in the variation vector generation condition DB 122B. The transfer function is provided to suit the degree of influence on an objective variable, which represents a characteristic of change in the value of factor data.

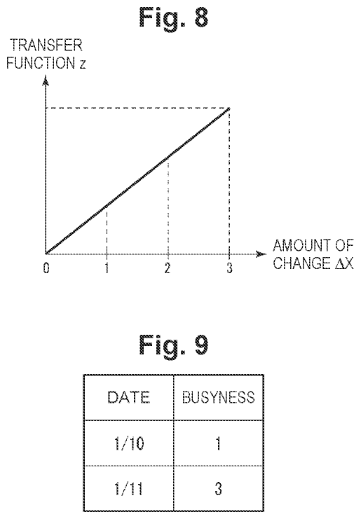

FIG. 8 is a diagram explaining a first example of a transfer function.

FIG. 8 shows a relationship between an amount of change ΔX in the value of factor data and the transfer function z. It is assumed here that Expressions (1) and (2) below hold, where n can be, for example, 1, 2, 3, . . . .

z=X′ (1)

X′=ΔX=X_{[n]}−X_{[n-1]} (2)

The interval/ordinal scale variation vector generation unit 23 generates a variation vector of factor data on an interval scale or an ordinal scale by referring to the factor data DB 121A, the variation vector generation condition DB 122B, and the interval/ordinal scale variation vector generation function DB 122C.

Details of procedures for generating a variation vector will be described below.

(a) The interval/ordinal scale variation vector generation unit 23 creates a vector structure based on variation vector generation conditions stored in the variation vector generation condition DB 122B.

FIG. 9 is a diagram showing, in tabular form, a first example of factor data on an interval scale, where the factor data is stored in the factor data DB 121A. Of factor data on an interval scale, FIG. 9 shows factor data on “busyness.”

For example, when the value of factor data on “busyness” whose scale type is interval scale is evaluation data that takes a value of 1 to 3 as shown in FIG. 9, the vector structure has a total of nine elements made up of three patterns (before change) by three patterns (after change).

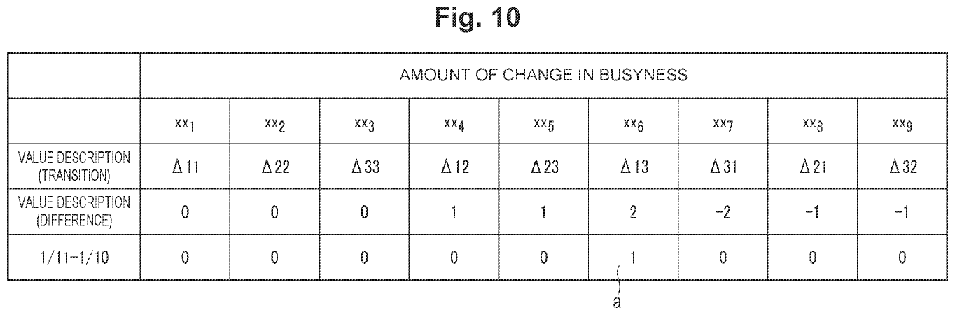

FIG. 10 is a diagram showing, in tabular form, a first example of variation vectors stored in the variation vector DB 121C. FIG. 10 shows variation vectors of factor data on “busyness.”

The columns xx₁ to xx₉ in FIG. 10 correspond to the number of elements in the vector structure.

(b) Based on the transfer function z stored in the interval/ordinal scale variation vector generation function DB 122C and using Expression (3) below, the interval/ordinal scale variation vector generation unit 23 calculates a corrected partial differential value Δx of a change in the value of factor data of an appropriate element in the created vector structure, e.g., an element concerning an amount of change Δ12 when the value changes from 1 to 2.

Δx=z_{(ΔX)} (3)

Δx: corrected partial differential value

ΔX: amount of change in factor

z: transfer function

Expression (3) above is used to calculate the amount of change in a factor between data on a predetermined date in a time series and data on a previous date, e.g., data on the previous day. However, a difference from a value k items ago in a time series or a difference from data a month earlier may be used depending on usage.

The interval/ordinal scale variation vector generation unit 23 normalizes (or standardizes) the calculated corrected partial differential values. Note that the values of irrelevant elements are set to 0. The values in the lowermost row of FIG. 10 correspond to the normalized corrected partial differential values.

Next, a first concrete example of calculation and normalization of corrected partial differential values will be described below.

By assuming that the amount of change and the impact on behavior are proportional to each other, the interval/ordinal scale variation vector generation unit 23 sets the transfer function to z=X′=ΔX.

If the factor data stored in the factor data DB 121A is as shown in FIG. 9, the corrected partial differential value of the amount of change in the value of factor data from the date 1/10 to the date 1/11 is given by Expression (4) below.

Δ13=z_{(ΔX)}=3−1=2 (4)

The interval/ordinal scale variation vector generation unit 23 normalizes corrected partial differential values by searching all the cells in the table shown in FIG. 10, the table concerning variation vectors stored in the variation vector DB 121C. The values in the lowermost row of FIG. 10 correspond to the normalized values.

In performing normalization, if the corrected partial differential has a maximum value larger than 1 (in the example of FIG. 10, “2” in Expression (4) of Δ13 above), the interval/ordinal scale variation vector generation unit 23 performs normalization using “1” (‘a’ in FIG. 10) as the maximum value, but if the maximum value of the corrected partial differential is equal to or smaller than 0, the interval/ordinal scale variation vector generation unit 23 performs normalization using “0” as the maximum value.

If the corrected partial differential has a minimum value smaller than 0, the interval/ordinal scale variation vector generation unit 23 performs normalization using “−1” as the minimum value, but if the minimum value of the corrected partial differential is equal to or larger than 0 (“0” in the example of FIG. 10), the interval/ordinal scale variation vector generation unit 23 performs normalization using “0” as the minimum value.

In principle, the corrected partial differential is normalized in the range of “−1” to “1,” but if the corrected partial differential has values only in a positive region, the corrected partial differential is normalized in the range of “0” to “1,” and if the corrected partial differential has values only in a negative region, the corrected partial differential is normalized in the range of “−1” to “0.”

Next, a second concrete example of calculation and normalization of corrected partial differential values will be described below.

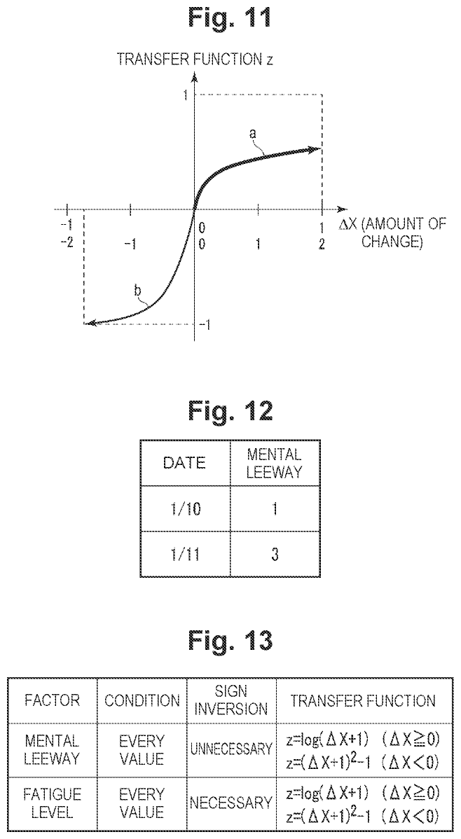

FIG. 11 is a diagram explaining a second example of the transfer function.

Here, by assuming that the characteristic of change (a degree of influence on objective variables) in the value of factor data has a relationship shown in FIG. 11, transfer functions given by Expressions (5) and (6) below are used. The abscissa in FIG. 11 represents plural examples of ΔX (the amount of change).

z=log(ΔX+1)(ΔX≥0) (5)

z=log(ΔX+1)²−1(ΔX<0) (6)

Of the transfer functions shown in FIG. 11, Expression (5) above is a transfer function (a in FIG. 11) used in changing in a positive direction.

Of the transfer functions shown in FIG. 11, Expression (6) above is a transfer function (b in FIG. 11) used in changing in a negative direction.

The transfer function used in changing in the positive direction reflects the following characteristics:

(a) a positive change, which has a lower subjective value than a negative change, has a small impact on behavior; and

(b) when the amount of change increases, the subjective value decreases rather than increasing in proportion.

The transfer function used in changing in the negative direction reflects the following characteristics:

(a) a negative change, which has a higher subjective value than a positive change, has a large impact on behavior; and

(b) when the amount of change increases, the subjective value decreases rather than increasing in proportion.

FIG. 12 is a diagram showing, in tabular form, a third example of the factor data stored in the factor data DB 121A. FIG. 12 shows factor data on “mental leeway” out of factor data on interval scales.

FIG. 13 is a diagram showing, in tabular form, a second example of the interval/ordinal scale variation vector generation functions stored in the interval/ordinal scale variation vector generation function DB 122C.

When the factor data shown in FIG. 12 is stored in the factor data DB 121A, and the interval/ordinal scale variation vector generation functions shown in FIG. 13 are stored in the interval/ordinal scale variation vector generation function DB 122C, the corrected partial differential value of the amount of change in the value of factor data from the date “1/10” to the date “1/11” is calculated, for example, as shown by Expression (7) below.

$$Δ13 = z ( Δ x ) = log ( ΔX + 1 ) = log ( 2 + 1 ) = 0.48 \qquad ( 7 )$$

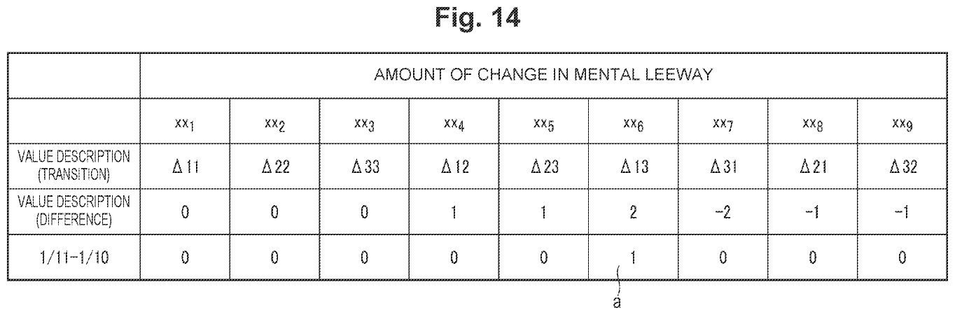

FIG. 14 is a diagram showing, in tabular form, a second example of the variation vectors stored in the variation vector DB 121C. FIG. 14 shows variation vectors of factor data on “mental leeway.”

In this example, the interval/ordinal scale variation vector generation unit 23 normalizes corrected partial differential values by searching all the cells in the table shown in FIG. 14, the table concerning variation vectors stored in the variation vector DB 121C. The values in the lowermost row of FIG. 14 correspond to the normalized corrected partial differential values.

In performing normalization, if the corrected partial differential has a maximum value larger than 1 (in the example of FIG. 14, “0.48” in Expression (7) of Δ13 above), the interval/ordinal scale variation vector generation unit 23 performs normalization using “1” (a in FIG. 14) as the maximum value, but if the maximum value of the corrected partial differential is equal to or smaller than 0, the interval/ordinal scale variation vector generation unit 23 performs normalization using “0” as the maximum value.

If the corrected partial differential has a minimum value smaller than 0, the interval/ordinal scale variation vector generation unit 23 performs normalization using “−1” as the minimum value, but if the minimum value of the corrected partial differential is equal to or larger than 0 (“0” in the example of FIG. 14), the interval/ordinal scale variation vector generation unit 23 performs normalization using “0” as the minimum value.

Next, a third concrete example of calculation and normalization of corrected partial differential values will be described below.

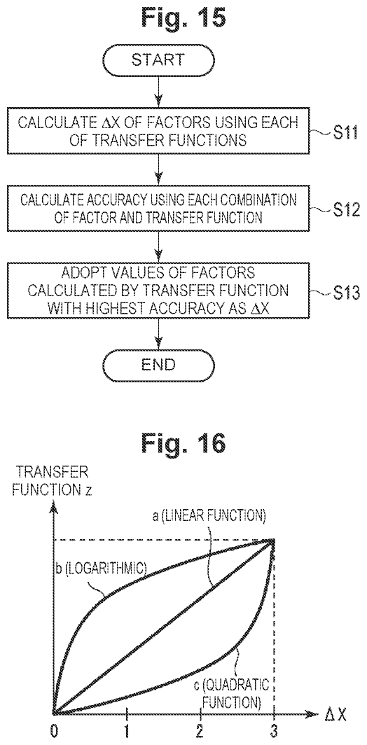

FIG. 15 is a flowchart showing an example of processing procedures for determining a corrected partial differential value.

FIG. 16 is a diagram showing an example of various transfer functions.

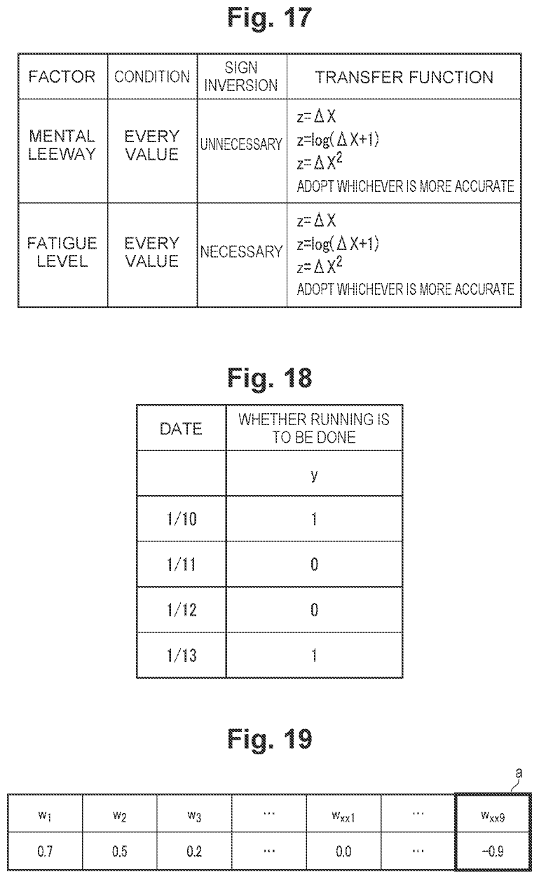

FIG. 17 is a diagram showing, in tabular form, a third example of the interval/ordinal scale variation vector generation functions stored in the interval/ordinal scale variation vector generation function DB 122C.

FIG. 16 shows transfer functions for factors, indicating that there are a linear function (a in FIG. 16), a logarithmic function (b in FIG. 16), and a quadratic function (c in FIG. 16) as candidates for a transfer function for use to calculate the corrected partial differential value Δx.

In this example, the interval/ordinal scale variation vector generation unit 23 calculates corrected partial differential values Δx of factors using each of the plural transfer functions, which are candidates for the transfer function for use to calculate the corrected partial differential value Δx (S11).

Using each combination of a factor and a transfer function, the interval/ordinal scale variation vector generation unit 23 compares Δx calculated in S11 with a correct answer acquired in advance and thereby calculates the accuracy of each transfer function (S12).

The interval/ordinal scale variation vector generation unit 23 selects the corrected partial differential value Δx calculated using the transfer function determined in S12 as having the highest accuracy (smallest error) and adopts (determines) the corrected partial differential value Δx as a final corrected partial differential value Δx (S13).

(4) Objective Variable Data Collection Unit

The objective variable data collection unit 24 collects values of an objective variable with specified timing (e.g., at a specified time or at the time when data is acquired) and registers the collected values of the objective variable in the objective variable DB 121D.

FIG. 18 is a diagram showing, in tabular form, an example of an objective variable stored in the objective variable DB 121D. If the objective variable stored in the objective variable DB 121D is, for example, “whether running is to be done,” a value y of the objective variable is, for example, as shown in FIG. 18, where the objective variable is collected data registered in the objective variable DB 121D.

(5) Regression Analysis Data Acquisition Unit

With specified timing or with desired timing of the user, the regression analysis data acquisition unit 25 acquires the explanatory variables (e.g., xᵢ (i: 1 to n) and xxⱼ (j: 1 to n)) needed for regression analysis and data of objective variable (e.g., y) from the one-hot vector DB 121B, the variation vector DB 121C, and the objective variable DB 121D and transmits the acquired data to the regression analyzer unit 26, where xᵢ indicates elements of the one-hot vector based on new input of factor data (i is the number of elements) and xxⱼ indicates elements of a variation vector based on the new input of the factor data (j is the number of elements).

(6) Regression Analyzer Unit

The regression analyzer unit 26 conducts regression analysis, such as multiple regression analysis or logistics regression analysis, which is regressive analysis of a relationship between an objective variable and an explanatory variable, based on the data received from the regression analysis data acquisition unit 25 and saves weights w calculated by the regression analysis in weight DB 121F.

If the accuracy of transfer functions are calculated in S12 above, the calculation results are stored in the generation function accuracy DB 121E by the regression analysis data acquisition unit 25 via the regression analyzer unit 26.

FIG. 19 is a diagram showing, in tabular form, an example of weights calculated by regression analysis and stored in the weight DB 121F.

From the value indicated by a in FIG. 19, it can be presumed that “a great reduction in fatigue of a very tired user greatly affects behavior of the user.”

(7) Weight Application Unit

As described above, regarding ordinal/interval scale data, if weights are calculated for the amounts of change as an explanatory variable, the weights can be used, for example, as follows.

(7-1) Use for Impact Scores that Represent Impacts in Various States of Motives/Disincentives

Regarding a factor that motivates user behavior and a factor assumed to deter user behavior, the weight application unit 27 uses a score representing the extent to which each state of the user affects the user behavior as a user-behavior impact score.

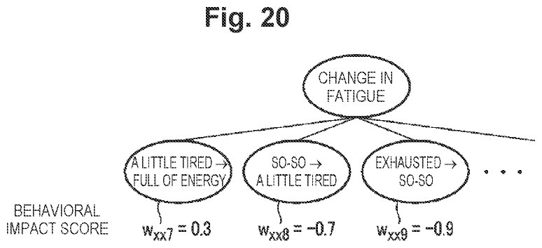

FIG. 20 is a diagram showing an example of behavioral impact scores.

In this way, factors that motivate user behavior and factors that deter user behavior can be calculated closely with higher accuracy.

(7-2) Feasibility Prediction

When data on factor data is newly acquired, the weight application unit 27 predicts the value of the objective variable based on Expression (8) below using weight information registered in the weight DB 121F. This makes it possible to calculate predictive values of the objective variable more accurately.

$$\begin{matrix}[ Math . 1 ] & \\ = {\sum }_{i = 1}^{n} {w}_{i} {x}_{i}^{′} + {\sum }_{j = 1}^{N} {w}_{j} {xx}_{j}^{′} & ( 8 )\end{matrix}$$

y′: objective variable to be predicted

wᵢ: weight of element of one-hot vector

wⱼ: weight of element of variation vector

x′ᵢ: explanatory variable (element of one-hot vector based on new input of factor data) used for prediction

xx′ⱼ: explanatory variable (element of variation vector based on new input of factor data) used for prediction

As described above, one embodiment of the present invention includes collecting factor data assumed to affect to-be-predicted data serving as an objective variable; and generating an explanatory variable for each type of the collected factor data based on a degree of influence of a factor on the objective variable, which represents a characteristic of a change in the value of the factor data. Thus, the embodiment of the present invention can improve accuracy of data analysis conducted using explanatory variables.

The techniques described in the above embodiments can be distributed as programs (software means) executable by a computer by being stored in a recording medium or by being transmitted via a communications medium, where examples of the recording medium include magnetic disks (a floppy (registered trademark) disk, a hard disk, and the like), optical disks (a CD-ROM, a DVD, an MO, and the like), semiconductor memories (a ROM, a RAM, a flush memory, and the like). Note that the programs stored in the medium also include a configuration program that configures, in the computer, software means (including not only execution programs, but also tables and data structures) to be executed by the computer. The computer that implements the present apparatus performs the above processes by reading the programs recorded on the recording medium by building software means in some cases using the configuration program, and by allowing the software means to control operation. Note that the recording medium referred to herein is not limited to distribution media, and includes storage media such as magnetic disks and semiconductor memories provided in the computer or devices connected via a network.

Note that the present invention is not limited to the above embodiments, and may be modified in various forms in the implementation stage without departing from the gist of the invention. The embodiments may be implemented in combination as appropriate, offering combined effects. Furthermore, the above embodiments include various inventions, and various inventions can be extracted through appropriate combinations of the disclosed components. For example, even if some of the components are removed from any of the embodiments, the resulting configuration can be extracted as an invention as long as the configuration can solve the problems and provide the advantages.

### REFERENCE SIGNS LIST

1 Contribution estimation apparatus
21 Factor data collection unit
22 One-hot vector generation unit
23 Interval/ordinal scale variation vector generation unit
24 Objective variable data collection unit
25 Regression analysis data acquisition unit
26 Regression analyzer unit
27 Weight application unit
121 Collection and generation DB
121A Factor data DB
121B One-hot vector DB
121C Variation vector DB
121D Objective variable DB
121E Generation function accuracy DB
121F Weight DB
122 Condition DB
122A One-hot vector generation condition DB
122B Variation vector generation condition DB
122C Interval/ordinal scale variation vector generation function DB

1. A data analysis apparatus comprising:
a processor; and
a storage medium having computer program instructions stored thereon, when executed by the processor, perform to:
collecting factor data assumed to affect to-be-predicted data serving as an objective variable; and
calculating a corrected partial differential value of a change in a value of the factor data based on a degree of influence of a factor on the objective variable, which is a characteristic of the change in the value of the factor data, for each type of the factor data collected and generating an explanatory variable based on the corrected partial differential value.

2. The data analysis apparatus according to claim 1, wherein the computer program instructions further perform to calculate the corrected partial differential value using transfer functions set according to the degree of influence of the factor on the objective variable.

3. The data analysis apparatus according to claim 2, wherein the computer program instructions further perform to calculate the corrected partial differential value using a transfer function that minimizes a deviation from correct data out of the set transfer functions.

4. The data analysis apparatus according to claim 1 wherein the computer program instructions further perform to
collecting values of the objective variable; and
regressively analyze a relationship between the objective variable and the explanatory variable.

5. A data analysis method performed by a data analysis apparatus, the method comprising:
collecting factor data assumed to affect to-be-predicted data serving as an objective variable; and
calculating a corrected partial differential value of a change in a value of the factor data based on a degree of influence of a factor on the objective variable, which represents a characteristic of the change in the value of the factor data, for each type of the collected factor data and generating an explanatory variable based on the corrected partial differential value.

6. The data analysis method according to claim 5, wherein the generating includes calculating the corrected partial differential value using transfer functions set according to the degree of influence of the factor on the objective variable.

7. The data analysis method according to claim 5, further comprising:
collecting values of the objective variable; and
regressively analyzing a relationship between the collected objective variable and the generated explanatory variable.

8. A non-transitory computer-readable medium having computer-executable instructions that, upon execution of the instructions by a processor of a computer, cause the computer to function as the data analysis apparatus according to claim 1.
